SEQUENCE LISTING (5 sequences)

SEQ ID NO: 1 - protein, 348 aa, Homo sapiens
MSDLGAVISLLLWGRQLFALYSGNDVTDISDDRFPKPPEIANGYVEHLFRYQCKNYYRLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYHQVDIGLIKLKQKVLVNERVMPICLPSKNYAEVGRVGYVSGWGQSDNFKLTDHLKYVMLPVADQYDCITHYEGSTCPKWKAPKSPVGVQPILNEHTFCVGMSKYQEDTCYGDAGSAFAVHDLEEDTWYAAGILSFDKSCAVAEYGVYVKVTSIQHWVQKTIAEN

SEQ ID NO: 2 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 3 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 4 - protein, 420 aa, Homo sapiens
MPHSTALPEARPTKMSALGAVIALLLWGQLFAVDSGNDVTDIADDGCPKPPEIAHGYVEHSVRYQCKNYYKLRTEGDGVYTLNNEKQWINKAVGDKLPECEADDGCPKPPEIAHGYVEHSVRYQCKNYYKLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 5 - protein, 343 aa, Homo sapiens
MDNQAERESEAGVGLQRDEDDAPLCEDVELQDGDLSPEEKIFLREFPRLKEDLKGNIDKLRALADDIDKTHKKFTKANMVATSTAVISGVMSLLGLALAPATGGGSLLLSTAGQGLATAAGVTSIVSGTLERSKNKEAQARAEDILPTYDQEDREDEEEKADYVTAAGKIIYNLRNTLKYAKKNVRAFWKLRANPRLANATKRLLTTGQVSSRSRVQVQKAFAGTTLAMTKNARVLGGVMSAFSLGYDLATLSKEWKHLKEGARTKFAEELRAKALELERKLTELTQLYKSLQQKVRSRARGVGKDLTGTCETEAYWKELREHVWMWLWLCVCLCVCVYVQFT

(19) United States
(12) Patent Application Publication
(10) Pub. No.: US 2012/0214169 A1
(43) Pub. Date: Aug. 23, 2012

(54) DIFFERENTIAL LEVELS OF HAPTOGLODIN ISOFORMS IN SMALL CELL LUNG CANCER

(75) Inventor: Ajit Bharti, West Roxbury, MA (US)

(73) Assignee: BOSTON MEDICAL CENTER CORPORATION, Boston, MA (US)

(21) Appl. No.: 13/401,212

(22) Filed: Feb. 21, 2012

Related U.S. Application Data
(60) Provisional application No. 61/445,149, filed on Feb. 22, 2011.

(51) Int. Cl. G01N 33/574; C12Q 1/68; C12M 1/34; G01N 27/62

(52) U.S. Cl. 435/6.12; 435/23; 436/501; 435/287.2; 422/69

(57) ABSTRACT

The invention is directed to protein or nucleic acid assays for diagnosis, prognosis and monitoring of lung cancers, in particular small cell lung cancer using biomarkers comprising haptoglobin isoforms, or fragments or variants thereof.

### CROSS-REFERENCE TO RELATED APPLICATIONS

The present application claims benefit under 35 U.S.C. §119(e) of U.S. provisional application Ser. No. 61/445,149, filed on Feb. 22, 2011, the content of which is herein incorporated by reference in its entirety.

### FIELD OF THE INVENTION

The present application is directed to assays for and methods of diagnosis, prognosis and monitoring of lung cancers, in particular small cell lung cancer (SCLC), using biomarkers such as haptoglobin isoforms or fragments or variants thereof.

### BACKGROUND

Lung cancer is one of the leading causes of cancer-related deaths for both men and women in the U.S., and similar trend are seen worldwide. Lung cancer includes small cell lung cancer (SCLC) and non-small cell lung cancer (NSCLC). The overall survival rate for patients with SCLC is low, less than 5% over five years for the majority of SCLC patients and less than 20%-25% over five years for approximately one-third of SCLC patients. The lack of early diagnosis is one of the primary reasons for this high mortality rate.

Haptoglobins are tetrameric proteins comprising typically two alpha and two beta chains that known to at least bind free hemoglobin released from erythrocytes with high affinity and thereby inhibit its oxidative activity. In some clinical settings, the haptoglobin assay has been used to screen for and monitor intravascular hemolytic anemia (Gupta et al., Advances in Hematology Vol. 2011 (2011), Article ID 389854, 4 pages). Other diseases that have been discussed in connection with haptoglobin include high altitude pulmonary edema (Ahmad Y, et al. Funct Integr Genomics, 2011 Sep.), diabetic retinopathy (Goldenberg-Cohen N, et al. Retina, 2011 Sep.), and sickle cell disease (Santos M N, et al. Genet Test Mol Biomarkers, 2011 Oct.).

Serum biomarkers for lung cancer have been evaluated in the hope of achieving early detection of the lung disease, improving diagnosis, and monitoring recurrence after treatment. Currently, biomarker specificity and early stage diagnostic value in clinical applications are limited. Therefore, there is a need to develop an improved strategies for SCLC diagnosis and treatment.

### SUMMARY OF THE INVENTION

The present invention provides assays for and methods of diagnosis, prognosis and monitoring of lung cancers, in particular small cell lung cancer (SCLC), using biomarkers that have been linked to SCLC. The assays and methods are, at least in part, based on a discovery that differentially altered expression patterns of certain biological markers are associated only with SCLC patients. The biological markers comprise at least one haptoglobin isoform or fragment or variant thereof, the presence or increased expression of which is shown herein to be associated with SCLC.

Accordingly, in one embodiment the invention provides an assay comprising determining a level of one or more haptoglobin isoform, or fragment or variant thereof, in a biological sample of a subject; and comparing the level of the haptoglobin isoform, or fragment or variant thereof, in the biological sample of the subject with a reference, wherein an elevated level of the haptoglobin isoform, or fragment or variant thereof, in the sample of the subject relative to the reference indicates the subject may have small cell lung cancer (SCLC).

In another embodiment, the invention provides an assay comprising determining the levels of one or more haptoglobin isoform, or fragment or variant thereof, in a first biological sample obtained from a subject at a first time point and a second biological sample taken from the subject at a second time point, comparing the haptoglobin isoform, or fragment or variant thereof level in the first biological sample to the level of haptoglobin isoform, or fragment or variant thereof in the second biological sample, wherein an increase in the level of the haptoglobin isoform, or fragment or variant thereof, in the second biological sample compared with the first biological sample indicates that the subject is at risk for developing small cell lung cancer (SCLC).

In another embodiment, the invention provides an assay comprising determining level of one or more haptoglobin isoform, or fragment or variant thereof, in a first biological sample obtained from a subject diagnosed with small cell lung carcinoma (SCLC) at a first time point and in a second biological sample obtained from the subject at a later time point, wherein the subject is exposed to a medical treatment or medication between the first and the later time point, comparing the level of one or more haptoglobin isoform, or fragment or variant thereof between the first and the later time point, wherein a decrease in the level of the haptoglobin isoform, or fragment or variant thereof, in the sample obtained at the later time point compared with the first time point indicates an effective medical treatment or medication for the subject for treating SCLC.

In some aspects of all the embodiments of the invention, the haptoglobin isoform, or fragment or variant comprises at least one β haptoglobin isoform.

In some aspects of all the embodiments of the invention, the haptoglobin isoform, or fragment or variant comprises at least one haptoglobin minor isoform.

In some aspects of all the embodiments of the invention, the haptoglobin isoform, or fragment or variant comprises at least two haptoglobin minor isoforms.

In some aspects of all the embodiments of the invention, the haptoglobin minor isoform has a molecular weight of about 43 kDA to 44 kDa, and a basic pI.

In some aspects of all the embodiments of the invention, the haptoglobin minor isoform has a molecular weight of about 40 kDa, and an acidic pI.

In some aspects of all the embodiments of the invention, the level of haptoglobin isoforms, or fragment or variant thereof, is measured by a protein-binding moiety, such as an antibody, which can be a polyclonal or a monoclonal antibody. In some aspects the antibody is an antibody that binds to the haptoglobin proteins listed in Table 2 or antigenic epitopes or fragments thereof.

In some aspects of all the embodiments of the invention, the level of the haptoglobin isoform, or fragment or variant thereof is determined using a nucleic acid, typically mRNA level using the nucleic acid sequences encoding the haptoglobin and haptoglobin related proteins.

In yet another embodiment, the invention provides an assay, comprising detecting the expression of one or more haptoglobin minor isoforms in a biological sample obtained from a subject, wherein the haptoglobin minor isoform comprises an isoform that has a molecular weight of about 40 kDa, and an acidic pI; wherein the expression of the an isoform that has a molecular weight of about 40 kDa, and an acidic pI in the biological sample indicates that the subject has small cell lung carcinoma (SCLC).

In yet another embodiment, the invention provides an assay, comprising enriching at least one or more haptoglobin minor isoforms in a biological sample obtained from a subject; detecting the expression of one or more haptoglobin minor isoforms in a biological sample of a subject, wherein the one or more haptoglobin minor isoforms comprises a haptoglobin minor isoform that has a molecular weight of about 40 kDa, and an acidic pI, wherein presence of the expression of the haptoglobin minor isoform that has a molecular weight of about 40 kDa, and an acidic pI indicates that the subject has small cell lung carcinoma.

In some aspects of all the embodiments of the invention, enriching haptoglobin isoforms comprises removing albumin, removing immunoglobulin, or removing both albumin and immunglobin from the biological sample prior to the stem of detecting.

In another embodiment, the invention provides a device or a system for obtaining data regarding a biological sample from a subject comprising: a determination module configured to yield detectable signal from an assay indicating the presence or level of one or more haptoglobin isoform, or fragment or variant thereof from the biological sample of a subject; and an output module for displaying an output content for a user.

In some aspects of all the embodiments of the device or a system, the device or a system may further comprise a sample collection unit to hold the biological sample; a storage module configured to store data output from the determination module; and/or a comparison assembly adapted to compare the data stored on the storage module with a control data, and to provide a retrieved data as the output content; and/or a communication module for transmitting data from the determination module to an external device to compare with control data, and to transmitting a retrieved data back from the external device to the device as the output content.

The invention also provides a kit comprising: at least one reagent having specific affinity to one or more haptoglobin isoform or fragment or variant thereof, such as one or more antibodies or one or more nucleic acids, such as primer pairs, in a suitable container which may comprise a lyophilized reagent or optionally contain a buffer or other reagents in the same or a different vial that are used in detection of the one or more haptoglobin isoform, or fragment or variant thereof; and instructions for carrying out the method of any one of claims 1 to 19. The reagents may include buffers, secondary antibodies, nucleic acid probes, labels enzymes and the like.

In some aspects of the kits, the kits further comprise a set of control values to the one or more haptoglobin isoform, or fragment or variant thereof; and/or positive and/or negative assay function controls, such as proteins or protein fragments or nucleic acids.

### BRIEF DESCRIPTION OF THE DRAWINGS

FIGS. 1A and 1B present data showing two dimensional gel electrophoresis and immunoblot analysis with anti-haptoglobin antibody against serum protein (total of 500 μg) from control (FIG. 1A) and SCLC patient (FIG. 1B). Serum proteins were first separated on pI 4-7 IPG strips and then on 12% SDS-PAGE. FIG. 1A shows that β, α-1 and α-2 haptoglobin chains were observed at 47 kDa, 9 kDa and 19 kDa, respectively, as indicated by arrows. FIG. 1B shows that all the above mentioned isoforms in control were present in patient samples, and a series of differentially expressed isoforms were also recorded. These isoforms were 5 kDa smaller than the 47 kDa β isoform, as shown in FIG. 1B, spot a, b, c and d.

FIGS. 2A and 2B present data showing the haptoglobin enrichment by removal of albumin and immunoglobulins. Serum samples from control and SCLC patients were ethanol-precipitated and immunoglobulin was removed by protein A affinity purification. Haptoglobin-enriched fractions were analyzed by SDS-PAGE and coomassie staining (FIG. 2A) and immunoblot analysis with anti-haptoglobin antibody (FIG. 2B). In each figure, lane 1 shows the albumin rich fraction; lane 2 shows the haptoglobin rich fraction; and lane 3 shows the unfractionated control serum.

FIGS. 3A and 3B present data relating to the characterization of haptoglobin isoforms by 2D-SDS-PAGE and MS. Haptoglobin-enriched serum from control (FIG. 3A) and SCLC patient (FIG. 3B) were analyzed by 2D-GE and SDS-PAGE. The proteins were detected by silver staining. Protein spots (circled and numbered) from both control and SCLC were processed by in-gel digestion. Trypsin digested proteins were analyzed by MALD-TOF-TOF-MS. Several proteins, including ten haptoglobin isoforms were characterized (see Table 1).

FIGS. 4A and 4B present data relating to the characterization of differentially present/expressed haptoglobin isoforms in SCLC patient samples: Control (FIG. 4A) and SCLC patient serum samples (FIG. 4B) were enriched for haptoglobin. Haptoglobin enriched samples were analyzed by 2D-GE-SDS-PAGE and silver staining. Two sets of proteins, one close to the j chain and the other between the a-1 and a-2 chains were differentially present in SCLC patient serum samples only (shown in insets).

FIG. 5 presents the representative MS data from a spot identified as haptoglobin. Trypsin digest of the 40 kDa negatively stained spot (from FIG. 4B, lower inset) was analyzed by MS. MS spectrum representing several haptoglobin peptides (marked by arrow) was identified as of haptoglobin by mass fingerprinting (FIG. 5A). One specific haptoglobin peptide with an m/z of 1439 was further analyzed to generate MS/MS data. The sequence analysis resulted in a significant y and b series representing haptoglobin peptide (FIG. 5B).

### DETAILED DESCRIPTION OF THE INVENTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include plural references and vice versa unless the context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below.

Other than in the operating examples, or where otherwise indicated, all numbers expressing quantities of ingredients or reaction conditions used herein should be understood as modified in all instances by the term “about.”

All patents and other publications identified in the specification, figures and examples are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The present invention provides for methods of diagnosis, prognosis and monitoring of lung cancers, in particular SCLC, using biomarkers that have been linked to SCLC. The methods are, at least in part, based on a discovery that differentially altered expression patterns of certain biological markers are associated with SCLC patients only. The markers comprise at least one of haptoglobin isoforms or fragments or variants, the presence or increased expression of which has been shown to be associated with SCLC.

Lung cancer, one of the leading causes of cancer-related death worldwide, is responsible for over one million deaths worldwide annually (WHO, Economics of Tobacco Control). A prediction from the American Cancer Society for 2009 is that 219,440 new cases of lung cancer will be diagnosed in the U.S., among which, 15% are small cell lung cancer (SCLC) and the rest are non-small cell lung cancer (NSCLC). Jemal et al., 56 CA Cancer J. Clin. 106-30 (2006). The overall survival rate for patients with SCLC is quite low. Approximately one-third of patients initially present with a limited disease (LD, within the chest cavity), and their survival rate is only 20-25% over five years. A majority of SCLC patients present with an extensive disease (ED, metastatic), however, and their chances of survival is less than 5% over five years. Current methods of detecting early stages of SCLC are poor and better strategies are needed.

Serum biomarkers for lung cancer have been studied in the hope of achieving early detection of the lung disease, improving diagnosis, predicting response, and/or monitoring recurrence after treatment. Gail et al., 80 J. Natl. Cancer Inst. 97-101 (1988). CEA, α-1-acid glycoprotein (AGP), neuron-specific enolase (NSE), chromogranin A (ChrA), bombesin-like gastrin-releasing peptide (GRP), and BB isoenzyme of creatine kinase (CKBB) are some of the candidate serum biomarkers for SCLC. Ma et al., 23 Anticancer Res. 49-62 (2003). Many potentially useful candidate serum biomarkers continue to be tested, particularly serum cytokines, e.g., vascular endothelial growth factor (VEGF), stem cell factor (SCF) and hepatocyte growth factor/scatter factor (HGF/SF). The present clinical usefulness of all these serum biomarkers, however, remains limited. McCarthy et al., 7 Cancer J. 175-7 (2001); Brundage, et al., 122 Chest 1037-57 (2002).

Haptoglobin is an acute phase protein and has been reported to be indicative of various pathological conditions including diverse forms of cancer, cirrhosis of the liver, and hepatitis-C. Ang et al., 5 J. Proteome Res. 2691-700 (2006); Okuyama et al., 118 Intl. J. Cancer 2803-08 (2006); Fujimura et al., 122 Intl. J. Cancer 39-49 (2008). Haptoglobin binds to hemoglobin and may function as a marker for hemolysis. Andersen et al., 164 Ann. Surg. 905-12 (1966). It has also been shown to inhibit prostaglandin synthesis and angiogenesis. Haptoglobin constitutes about 0.4%-2.6% of total serum protein. Langlois et al., 42 Clin. Chem. 1722-23 (1996); He et al., 343 Biochem. Biophys. Res. Commun. 496-503 (2006). Native haptoglobin is a hetero-tetramer composed of two α and two β subunits attached by disulfide bridges. Kurosky et al., 77 P.N.A.S. 3388-92 (1980). The human β subunit is a 38 kDa polypeptide linked to a isoforms by a disulfide bridge. The α subunit is represented by two isoforms, α-1 and α-2, while the β subunit has only one type. The amino acid sequences in a isoforms are similar: the α-1 isoform has 83 amino acids (9 kDa polypeptide); and the α-2 isoform is a duplicate of the α-1 chain, with a repeat insertion of amino acid residues 12 to 70, leading to 142 amino acids (20 kDa polypeptide). Different from α subunits, β-haptoglobin has a high level of glycosylation. The β-haptoglobin chain has 243 amino acids, with a molecular mass of more than 40 kDa. It is estimated that the β chain contains approximately 30% carbohydrate moiety. Maeda et al., 83 P.N.A.S. 73950-59 (1986); Patzelt et al., 9 Electrophoresis 393-97 (1988).

Haptoglobin has at least six possible phenotypes. The six phenotypes, in combination with two post translational modifications, glycosylation and deamidation, lead to large numbers of possible haptoglobin isoforms. Recent studies suggest a possible correlation between specific haptoglobin glycosylation and particular disease conditions. It has been reported that SCLC patients exhibit a higher circulatory level of a-haptoglobin. A possible correlation between the disease state and the level of a-haptoglobin in serum was also indicated. Bharti et al., 24 Anticancer Res. 1031-38 (2004).

In the present invention, serum samples and particularly the haptoglobin-enriched serum fractions of both control (i.e., disease-free subjects) and SCLC patients were analyzed to identify the differential expression of specific haptoglobin isoforms in SCLC patient serum. The samples were analyzed by two dimensional gel electrophoresis (2D-GE) and sodium dodecyl sulfate polyacrylamide gel electrophoresis (SDS PAGE). Once fractionated, the proteins were characterized either by immunoblotting or silver stain combined with mass spectrometry. The present invention shows that higher serum levels of both α- and β-haptoglobin isoforms were observed in SCLC patient samples. Additionally, the present invention shows that a β-chain variant (5 kDa smaller than full length) of haptoglobin was expressed solely in SCLC patients.

Accordingly, in one aspect, the present invention provides for a method of detecting the likelihood of SCLC in a subject. The method comprises determining a level of at least one haptoglobin isoform, or fragment or variant thereof, in a biological sample of a subject; and comparing the level of the haptoglobin isoform, or fragment or variant thereof, with the level of the haptoglobin isoform, or fragment or variant thereof, in a control, wherein an elevated level of the haptoglobin isoform, or fragment or variant thereof, in the sample of the subject relative to the control indicates that the subject may have SCLC.

In another aspect, the present invention provides for a method of monitoring a subject at risk for developing SCLC. The method comprises the steps of obtaining a first biological sample from a subject at a first time point and a second biological sample at a later time point; and determining the levels of at least one haptoglobin isoform, or fragment or variant thereof, in the first and second biological samples, wherein an increase in the level of the haptoglobin isoform, or fragment or variant thereof, in the second biological sample compared with the first biological sample indicates that the subject has the risk of developing SCLC.

In another aspect, the present invention provides for a method of monitoring the efficacy of a medical treatment or a medication on a subject being treated for SCLC. The method comprises obtaining a first biological sample from a subject at a first time point and a second biological sample at a later time point, wherein the subject is exposed to a medical treatment or medication on or after the earlier time point; and determining the levels of at least one haptoglobin isoform, or fragment or variant thereof, in the first and second biological samples, wherein a decrease the level of the haptoglobin isoform, or fragment or variant thereof, in the second biological sample compared with the first biological sample indicates an effective medical treatment or medication on the subject for treating SCLC.

The terms “patient”, “subject” and “individual” are used interchangeably herein, and include humans and non-human subjects. Non-human subjects may include non-human primates (particularly higher primates), sheep, goat, horse, cows, pig, guinea pig, rodents (e.g., mouse or rat), cat, rabbits, sports animals, and non-mammals such as chickens, amphibians, reptiles, etc. In some embodiments, the subject is a human subject. In other embodiments, the subject is a non-human, such as an experimental animal or animal-substitute as a disease model, or a domestic animal or a farm animal.

As used herein, the term “biological sample” may refer to a cell or population of cells or a quantity of tissue or fluid from a subject. Most often, the sample has been removed from a subject, but the term “biological sample” can also refer to cells or tissue analyzed in vivo, i.e., without removal from the subject. Biological sample may be a sample of tissue or fluid isolated from an individual, including but not limited to, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, samples of in vitro cell culture constituent, and ex vivo cultivated tissues. In some embodiments, a biological sample is from a resection, bronchoscopic biopsy, or core needle biopsy of a primary, secondary or metastatic tumor, or a cellblock from pleural fluid. In addition, fine needle aspirate biological samples are also useful. In some embodiments, a biological sample is primary ascite cells. Samples can be fresh, frozen, fixed, or optionally paraffin-embedded, or subjected to other tissue preservation methods.

A biological sample can mean a sample of biological tissue or fluid that comprises protein or cells. Such samples include, but are not limited to, tissue isolated from subjects or animals. Biological samples may also include sections of tissues such as biopsy and autopsy samples, frozen sections taken for histological purposes, blood, plasma, serum, sputum, stool, tears, mucus, hair, and skin. Biological samples also include explants and primary and/or transformed cell cultures derived from patient tissues. A biological sample may be provided by removing a sample of cells from subject, but can also be accomplished by using previously isolated cells (e.g., isolated by another person, at another time, and/or for another purpose), or by performing the methods of the invention in vivo. Archival tissues, such as those having treatment or outcome history may also be used.

The definition of biological sample also includes samples that have been manipulated in any way after their procurement, such as by treatment with reagents, solubilization, or enrichment for certain components, such as proteins or polynucleotides. In one embodiment, the abundance of the marker in the biological sample is low, the sample manipulation thus include enrich the markers. For example, to detect the differential expression of some haptoglobin minor isoforms, the content of haptoglobin in control and patient serum samples may be enriched. Enrichment of haptoglobin may be achieved by removing albumin, removing immunoglobulin from the samples, or combinations of these removing steps.

Acute phase proteins, such as haptoglobin has been shown to be significantly higher in the sera of patients with inflammatory diseases and cancer. Turner et al., 376 Adv. Exp. Med. Biol. 231-38 (1995); Thompson et al., 56 Br. J. Cancer 605-10 (1987); Boise et al., 74 Cell 597-608 (1993). This may be due to the epithelial-mesenchymal transition phenomenon, and the fact that tumor tissue demonstrates similarities with inflammatory cells, particularly activated fibroblasts. Hey et al., 10 Semin. Fetal. Neonatal. Med. 377-87 (2005); Lee et al., 151 Arch. Virol. 1651-58 (2006). Although haptoglobin is synthesized in the liver, higher levels of haptoglobin have been observed in tumor tissues. Ahmed et al., 91 Br. J. Cancer 129-40 (2004). Several studies have reported a higher level of serum haptoglobin in a variety of cancers including: ovarian, breast, small cell lung, pancreatic, liver and prostate cancers. Ang et al., 2006; Okuyama et al., 2006; Fujimura et al., 2008; Bharti et al., 2004; Thompson et al., 1987; Boise et al., 1993; Ahmed et al., 2004; Kossowska et al., 43 Clin. Chem. Lab Med. 361-69 (2005). The convergent themes of these studies are a higher circulatory level of serum haptoglobin and an altered glycosylation status of the β-chain.

Higher levels of haptoglobin may be attributed to higher levels of the protein. The increased glycosylation of the β-isoform, however, has been considered the major reason of higher level of serum haptoglobin in cancer patients. A correlation exists between cancer progression and the glycosylation of haptoglobin isoforms for patients with ovarian cancer: changes in glycoforms in haptoglobin β-chain, α1-acid glycoprotein, and α1-antichymotrypsin lead to changes in the total serum glycome of patients with advanced ovarian cancer. These changes include increases in levels of core fucosylation, agalactosyl biantennary glycans (FA2) and sialyl Lewis x (SLe x). Tabares et al., 16 Glycobiol. 132-45 (2006).

As noted, haptoglobin has at least six possible phenotypes. The six phenotypes, in combination with two post translational modifications, glycosylation and deamidation, lead to large numbers of possible haptoglobin isoforms. So far, there has been no discovery regarding the specificity of differential expression of haptoglobin isoforms or specific glycans in SCLC subjects. The present invention, however, shows surprisingly the differential expression of minor isoforms of the β chain between control (i.e., healthy subject) sera and SCLC patient sera.

Accordingly, in some embodiments, the haptoglobin isoform, fragment or variant thereof, for diagnosis, prognosis, and monitoring of lung cancer such as SCLC comprises at least one β-haptoglobin isoform.

In other embodiments, the haptoglobin isoform, fragment or variant thereof comprises at least one haptoglobin minor isoform. The haptoglobin minor isoform may refer to any fragments or variants of α or β-chain haptoglobin that may have smaller abundance than the major α or β haptoglobin isoforms. For example, one set of haptoglobin minor isoform may be about 1 kDa to 2 kDa below the 45 kDa β isoform, and has a basic pIs between 5.5 and 7.0. Another set of haptoglobin minor isoforms includes 3 to 5 isoforms (e.g., 4 isoforms) that are about 5 kDa smaller than the 45 kDa β isoform, and have acidic pIs between 4.5 and 6.0. The 40 kDa minor β haptoglobin isoforms were observed only in SCLC patient sera, not in the control sera, and have not yet been reported in any other cancer or inflammatory disease. Two or more of these haptoglobin isoforms may be used in the method of the present invention.

In some aspects of all the embodiments of the invention, the haptoglobin binding moiety, such as an antibody, is directed to SEQ ID NO: 1; SEQ ID NO: 2; or SEQ ID NO: 4 or any combination thereof or antigenic/immunogenic fragments/epitopes thereof to allow detection of the haptoglobin minor isoforms set forth above. In some aspects of all the embodiments of the invention, the haptoglobin binding moiety is directed to SEQ ID NO: 2 or antigenic/immunogenic fragments/epitopes thereof. In some aspects of all the embodiments of the invention, the haptoglobin binding moiety is directed to SEQ ID NO: 4 or antigenic/immunogenic fragments/epitopes thereof. In some aspects of all the embodiments of the invention, the haptoglobin binding moiety is directed to SEQ ID NO: 2 and 4 or antigenic/immunogenic fragments/epitopes thereof. In some aspects of all the embodiments of the invention, the haptoglobin binding moiety is directed to SEQ ID NO: 1 alone or in combination with SEQ ID NO: 2 and/or SEQ ID NO: 4 or antigenic/immunogenic fragments/epitopes thereof.

In some embodiments, to determine the differential expression of haptoglobin isoforms, control and SCLC patient serum samples were analyzed by 2D-GE. The fractionated proteins were either characterized by silver stain and MS analysis or by immunoblot analysis with anti-haptoglobin antibody. Comparison of control and SCLC patients by 2D-GE and immunoblot analysis indicated that: both the β and α haptoglobin isoforms were elevated in patient sera; and surprisingly, β chain minor isoforms were differentially expressed between control and SCLC patient. Thus, we discovered that the β chain minor isoforms can be used to differentiate patients who have SCLC as opposed to other type of lung cancer.

Additionally, the heterogeneous pattern of α chain was similar in control sera and in SCLC patient sera. Characterization of haptoglobin by 2D-GE and MS-based protein methods has demonstrated that the mobility shift in α-1 and α-2 isoforms are due to the changes in amino acids leading to different charge states. Connell et al., 193 Nature 505-6 (1962); Brune et al., 12 Nucleic Acids Res. 4531-38 (1984).

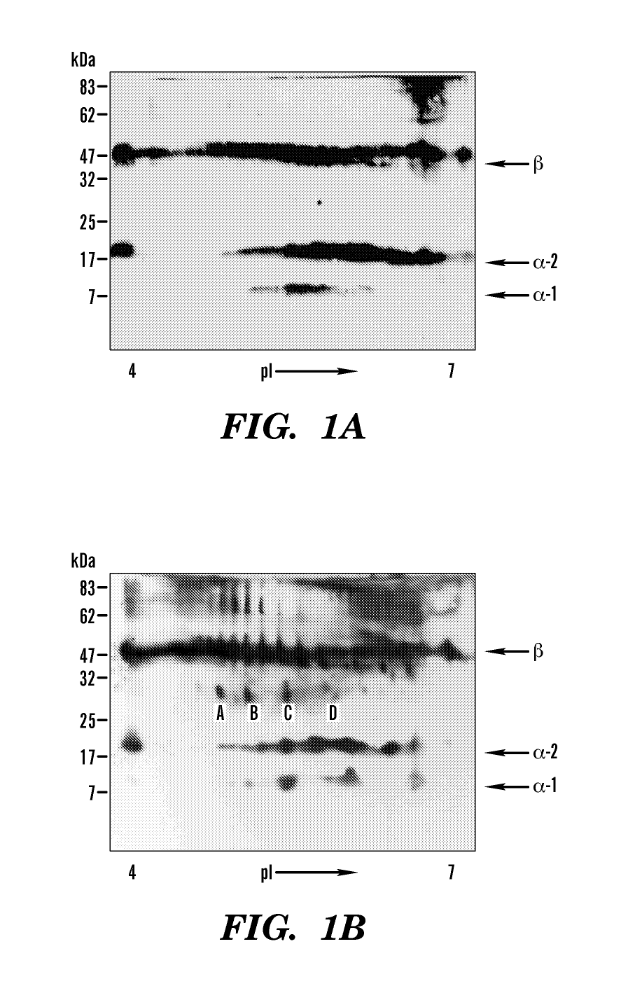

Surprisingly, differential expression of minor isoforms of the β chain exists between control sera and SCLC patient sera. The data from 2D-GE and immunoblots showed two different sets of spots: One set included several minor isoforms that are about 1 kDa to 2 kDa below the 45 kDa β isoform (herein referred to as the 44 kDa isoform), having basic pIs between 5.5 and 7.0. The other set included 3 to 5 isoforms (e.g., 4 isoforms) that are about 5 kDa smaller than the 45 kDa β isoform (herein referred to as the 40 kDa isoforms), having acidic pIs between 4.5 and 6.0. The 40 kDa isoforms were observed only in SCLC patient samples (FIGS. 1A and 1B).

In SCLC patient serum samples, several 43 kDA to 44 kDa minor isoforms with altered mobility towards basic pIs were observed. These isoforms may correlate to SCLC disease progression. The 44 kDa minor β haptoglobin isoforms appeared in doublet and have also been observed in alcoholic and cirrhotic patients. Gravel et al., 220 Biochem. Biophys. Res. Commun. 78-85 (1996). These changes have been attributed to altered glycosylation. The loss of terminal sialic acid, the galactose residue and the N-acetylglucosamine residue which are located at the penultimate and antepenultimate position of the glycan chains, respectively. Although the sialic acid content of major isoforms determines their separation by isoelectric focusing, however, the analysis of doublets of minor isoforms indicated a similar glycan structure, thus attributing the heterogeneity in minor isoforms to modifications other than N-glycan structure. He et al., 2006.

The 40 kDa minor β haptoglobin isoforms were observed only in SCLC patient sera, not in the control sera. This set of haptoglobin isoforms has not yet been reported in any other cancer or inflammatory disease. The relative abundance of these isoforms was very low. To further characterize these isoforms, control and SCLC patient serum samples were enriched with haptoglobin by removing albumin and immunoglobulins. Albumin was removed by ethanol precipitation as described. Colantonio et al., 5 Proteomics 3831-35 (2005). In this procedure, removal of highly abundant proteins such as albumin is coupled to powerful protein separation methods in order to increase sample loads, thus facilitating detection and identification of low abundance proteins. The haptoglobin-enriched serum fractions from control and SCLC patients were analyzed by 2D-GE and silver staining. Two 40 kDa protein spots were visible only in SCLC patient sera. These spots were negatively stained indicating the acidic nature of the proteins. Goldberg et al., 251 Anal. Biochem. 227-33 (1997). One of the spots was identified as haptoglobin, while the other was identified as apolypoprotein. The other two minor haptoglobins identified by immunoblotting were not observed in silver stain gels, possibly due to the lack of comparative sensitivity of silver staining.

Serum haptoglobin has been characterized to determine the correlation between glycosylation status and prostate cancer progression. Fujimura et al., 2008. Haptoglobin was purified from control, benign and prostate cancer patients and characterized the glycans by mass spectrometry. The conclusion was that levels of haptoglobin are enhanced in sera of prostate cancer patients, and the N-glycans attached to a defined region of its β chain are characterized by enhanced branching as well as antenna fucosylation. Fujimura et al., 2008. The putative glycan structure was further characterized, relative abundance of the putative glycan structure was determined. Most of the glycans, except three, were increased in patient sera. Correspondingly, the reduction in the level of these three glycans was also demonstrated in patient sera, and the mass of these glycans varied from 5 kDa to 6 kDa.

These 40 kDa minor haptoglobin isoforms may also be characterized to address if the reduction in certain glycans in SCLC patient serum attribute to differential expression of the minor β haptoglobin isoforms in patient sera. Deletion of these glycans will significantly change the pI to basic, however, thus the loss of a glycan structure does not explain the differential expression of these minor β haptoglobin isoforms.

These minor isoforms observed in SCLC patient are recognized by anti-haptoglobin antibody and by mass spectrometry. The anti-haptoglobin antibodies are highly specific to haptoglobin proteins as they typically have little or no cross reactivity with the most abundant proteins, i.e., albumin. In addition, at least one minor isoform was characterized by MS analysis and was identified as haptoglobin isoform. Therefore, these differentially expressed proteins are identified to be haptoglobin isoforms.

Accordingly, one aspect of the invention provides for a method of detecting SCLC in a subject. The method comprises detecting the expression of one or more haptoglobin minor isoforms in a biological sample of a subject, wherein the haptoglobin minor isoform has a molecular weight of about 40 kDa, and an acidic pI, wherein the expression of the one or more haptoglobin isoforms in the biological sample of the subject indicates that the subject has SCLC. In some embodiment, the abundance of the one or more haptoglobin minor isoforms may be low. Hence the method may further comprise enriching one or more haptoglobin minor isoforms in the biological sample of the subject before detecting the expression of these haptoglobin isoforms.

Commonly, protein levels in a biological sample may be measured using an affinity-based measurement technology. Affinity-based measurement technology utilizes a molecule that specifically binds to the marker being measured (an “affinity reagent,” such as an antibody or aptamer). In one embodiment, the affinity-based measurement technology is a protein-binding based technology. The protein binding moiety may include, but are not limited to antibodies, recombinant antibodies, chimeric antibodies, tribodies, midibodies, protein-binding agents, small molecule, recombinant protein, peptides, aptamers, avimers and derivatives or fragments thereof. For example, the protein binding moiety may be an antibody, such as a polyclonal or a monoclonal anti-haptoglobin antibody.

The affinity-based technologies may include, but are not limited to, antibody-based assays (immunoassays) and assays utilizing aptamers (nucleic acid molecules which specifically bind to other molecules), such as ELONA. Additionally, assays utilizing both antibodies and aptamers are also contemplated (e.g., a sandwich format assay utilizing an antibody for capture and an aptamer for detection).

Immunoassay technology may include any immunoassay technology which can quantitatively or qualitatively measure the level of a haptoglobin isoform, fragment or variant thereof in a biological sample. Suitable immunoassay technology includes, but not limited to immunohistochemical analysis, radioimmunoassay (RIA), immunoradiometric assays, immunofluorescent assay, enzyme immunoassay, chemiluminescent assay, enzyme-linked immunosorbent assay (ELISA), sandwich immunoassays, gel diffusion precipitation reactions, immunodiffusion assays, in situ immunoassays, precipitation reactions, agglutination assays, complement fixation assays, isoform-specific chemical or enzymatic cleavage, protein array, protein A assays, immunoelectrophoresis assays, immuno-PCR, western blot assay, and the like. See, e.g., Ausubel et al., 1 CURRENT PROTOCOLS MOL. BIOL. (John Wiley & Sons, Inc., NY, 1994). Likewise, aptamer-based assays which can quantitatively or qualitatively measure the level of a haptoglobin isoform, fragment or variant thereof in a biological sample may be used in the methods of the invention. Generally, aptamers may be substituted for antibodies in nearly all formats of immunoassay, although aptamers allow additional assay formats (such as amplification of bound aptamers using nucleic acid amplification technology such as PCR (U.S. Pat. No. 4,683,202) or isothermal amplification with composite primers (U.S. Pat. Nos. 6,251,639 and 6,692,918).

A wide variety of affinity-based assays are known in the art. Affinity-based assays will utilize at least one epitope derived from the molecule that of interest, and many affinity-based assay formats utilize more than one epitope (e.g., two or more epitopes are involved in “sandwich” format assays; at least one epitope is used to capture the molecule, and at least one different epitope is used to detect the molecule).

Affinity-based assays may be in competition or direct reaction formats, utilize sandwich-type formats, and may further be heterogeneous (e.g., utilize solid supports) or homogenous (e.g., take place in a single phase) and/or utilize or immunoprecipitation. Many assays involve the use of labeled affinity reagent (e.g., antibody, polypeptide, or aptamer); the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA and ELONA assays.

The protein levels in a biological sample may also be measured through other technologies, such as spectroscopy-based technologies (e.g., matrix-assisted laser desorption ionization-time of flight, or MALDI-TOF, Mass Spectroscopy) or assays measuring bioactivity (e.g., assays measuring mitogenicity of growth factors).

The level of haptoglobin isoforms, fragments and variants in the control and patient samples may also be determined based on gel electrophoresis techniques, in particular SDS-PAGE, for instance, two dimensional PAGE (2D-PAGE), or two dimensional SDS-PAGE (2D-SDS-PAGE). In certain embodiments, the gel electrophoresis assay may use immobilized pH gradients (IPGs) with a pH ranging from about 4 to about 9, or from about 4 or about 7.

In certain embodiments of the present invention, one or more of the above mentioned techniques for measuring levels of haptoglobin isoforms, fragments and variants thereof, in the biological sample may be combined together, and may be further combined with other protein separation methods, particularly methods known to those skilled in the art, e.g., chromatography, size exclusion, and/or other protein precipitation techniques.

The methods described herein may be implemented using any device capable of implementing the methods. Examples of devices that may be used include but are not limited to electronic computational devices, including computers of all types. When the methods described in the present invention are implemented in a computer, the computer program that may be used to configure the computer to carry out the steps of the methods may be contained in any computer readable medium capable of containing the computer program. Examples of computer readable medium that may be used include but are not limited to diskettes, CDROMs, DVDs, ROM, RAM, and other memory and computer storage devices. The computer program that may be used to configure the computer to carry out the steps of the methods may also be provided over an electronic network, for example, over the internet, world wide web, an intranet, or other network.

In one example, the methods described in the present invention may be implemented in a system comprising a processor and a computer readable medium that includes program code means for causing the system to carry out the steps of the methods described in the present invention. The processor may be any processor capable of carrying out the operations needed for implementation of the methods. The program code means may be any code that when implemented in the system can cause the system to carry out the steps of the methods described in the present invention. Examples of program code means include but are not limited to instructions to carry out the methods described in this patent written in a high level computer language such as C++, Java, or Fortran; instructions to carry out the methods described in the present invention written in a low level computer language such as assembly language; or instructions to carry out the methods described in the present invention in a computer executable form such as compiled and linked machine language.

As will be understood by those of skill in the art, the mode of detection of the signal will depend on the detection system utilized in the assay. For example, if a radiolabeled detection reagent is utilized, the signal will be measured using a technology capable of quantitating the signal from the biological sample or of comparing the signal from the biological sample with the signal from a control sample, such as scintillation counting, autoradiography (typically combined with scanning densitometry), and the like. If a chemiluminescent detection system is used, then the signal will typically be detected using a luminometer. Methods for detecting signal from detection systems are well known in the art and need not be further described here.

When more than one haptoglobin isoforms are measured, the biological sample may be divided into a number of aliquots, with separate aliquots used to measure different haptoglobin isoforms (although division of the biological sample into multiple aliquots to allow multiple determinations of the levels of the haptoglobin isoforms in a particular sample are also contemplated). Alternately the biological sample (or an aliquot therefrom) may be tested to determine the levels of multiple haptoglobin isoforms in a single reaction using an assay capable of measuring the individual levels of different haptoglobin isoform in a single assay, such as an array-type assay or assay utilizing multiplexed detection technology (e.g., an assay utilizing detection reagents labeled with different fluorescent dye markers).

It is common in the art to perform “replicate” measurements when measuring levels of haptoglobin isoforms. Replicate measurements are ordinarily obtained by splitting a sample into multiple aliquots, and separately measuring the haptoglobin isoform(s) in separate reactions of the same assay system. Replicate measurements are not necessary to the methods of the invention, but many embodiments of the invention will utilize replicate testing, particularly duplicate and triplicate testing.

The process of comparing a level of one or more haptoglobin isoform, fragment or variant thereof from a subject and a control can be carried out in any convenient manner appropriate to the type of the value from the subject and control value for the haptoglobin isoform at issue. Generally, values measured in the methods of the invention may be quantitative values (e.g., quantitative values of concentration, such as nanograms of the molecule per milliliter of sample, or an absolute amount). Alternatively, values measured can be qualitative depending on the measurement techniques, and thus the mode of comparing a value from a subject and a control value can vary depending on the measurement technology employed. For example, the comparison can be made by inspecting the numerical data, by inspecting representations of the data (e.g., inspecting graphical representations such as bar or line graphs). In one example, when a qualitative calorimetric assay is used to measure levels of haptoglobin isoforms, the levels may be compared by visually comparing the intensity of the colored reaction product, by comparing data from densitometric or spectrometric measurements of the colored reaction product (e.g., comparing numerical data or graphical data, such as bar charts, derived from the measuring device), or by comparing density of bands or area of peaks from spectrometric measurements or assays.

As described herein, haptoglobin isoform in biological samples may be measured quantitatively (absolute values) or qualitatively (relative values). The respective levels of haptoglobin isoforms for a given assessment may or may not overlap. In some embodiments, quantitative values of particular haptoglobin isoform(s) in the biological samples may indicate a given level of SCLC progression. Hence quantitative values, such as concentrations of haptoglobin isoforms, can be used to compare the concentration of a haptoglobin isoform level from a subject to a control concentration of the haptoglobin isoform to diagnosis and/or monitor the progress of SCLC in patients.

In certain embodiments, the comparison is performed to determine the magnitude of the difference between the values from a subject and control values (e.g., comparing the “fold” or percentage difference between the value from a subject and the control value). A fold difference that is about equal to or greater than the minimum fold difference disclosed herein suggests or indicates a diagnosis of SCLC, or progression from mild stage of cancer to moderate stage of cancer, or vise versa when undergoing certain medication or medical treatment. A fold difference can be determined by measuring the absolute concentration of a haptoglobin isoform and comparing that to the absolute value of a control, or a fold difference can be measured by the relative difference between a control value and a sample value, where neither value is a measure of absolute concentration, and/or where both values are measured simultaneously. For example, an antibody array measures the relative concentration from which a fold change of haptoglobin isoform(s) may be determined. Accordingly, the magnitude of the difference between the measured value and the control value that suggests or indicates a particular diagnosis will depend on the particular haptoglobin isoform being measured to produce the measured value and the control value used (which in turn depends on the method being practiced).

The process of comparing may be manual (such as visual inspection by the practitioner of the method) or it may be automated. For example, an assay device (such as a luminometer for measuring chemiluminescent signals) may include circuitry and software enabling it to compare a value from a subject with a control value for a haptoglobin isoform. Alternately, a separate device (e.g., a digital computer) may be used to compare the value(s) from subject(s) and the control value(s). Automated devices for comparison may include stored control values for the haptoglobin isoform(s) being measured, or they may compare the value(s) from subject(s) with control values that are derived from contemporaneously measured control samples.

Provided herein are also kits and devices for carrying out any of the methods described herein. Kits of the present invention may comprise at least one reagent specific to at least one of the haptoglobin isoforms, fragments or variants thereof, and may further include instructions for carrying out a method of diagnosis, prognosis and monitoring of SCLC, as described herein.

In some embodiments, the kit comprises at least two or more different haptoglobin isoform-specific affinity reagents, where each reagent is specific for a different haptoglobin isoform. In some embodiments, the reagent(s) specific for a haptoglobin is an affinity reagent.

Kits comprising a single reagent specific for a haptoglobin may have the reagent enclosed in a container (e.g., a vial, ampoule, or other suitable storage container). Alternatively, the reagent may be bound to a substrate (e.g., an inner surface of an assay reaction vessel). Likewise, kits including more than one reagent may also have the reagents in containers (separately or in a mixture) or may have the reagents bound to a substrate.

Thus, in some embodiments, the kit further comprises at least one solid support wherein the reagent specific to at least one haptoglobin isoform, fragments or variants thereof is deposited on the support. In some examples, the solid support is in the format of a dipstick, a test strip, a latex bead, a microsphere or a multi-well plate.

In some embodiments, the haptoglobin-specific reagent(s) may be labeled with a detectable marker (such as a fluorescent dye or a detectable enzyme), or may be modified to facilitate detection (e.g., biotinylated to allow for detection with an avidin- or streptavidin-based detection system). In other embodiments, the haptoglobin-specific reagent may not be directly labeled or modified.

In certain embodiments, kits may also include one or more agents for detection of bound haptoglobin specific reagent. Detection agents and detection systems are those known in the art. For example, detection agents may include antibodies specific for the haptoglobin-specific reagent (e.g., secondary antibodies), primers for amplification of a haptoglobin-specific reagent that is nucleotide based (e.g., aptamer) or of a nucleotide ‘tag’ attached to the haptoglobin-specific reagent, avidin- or streptavidin-conjugates for detection of biotin-modified haptoglobin-specific reagent(s), and the like.

A modified substrate or other system for capture of haptoglobin isoforms or variants thereof may also be included in the kits of the invention, particularly when the kit is designed for use in a sandwich-format assay. The capture system may be any capture system useful in a haptoglobin assay system, as known in the art, such as a multi-well plate coated with a haptoglobin specific reagent, beads coated with a haptoglobin-specific reagent, and the like.

The instructions in the kit relating to the use of the kit for carrying out the invention generally describe how the contents of the kit are used to carry out the methods of the invention. Instructions may include information as sample requirements (e.g., form, pre-assay processing, and size), steps necessary to measure the marker(s) (i.e., haptoglobin isoforms, fragments, or variants), interpretation of results, and the like. Instructions supplied in the kits may include written instructions on a label or package insert (e.g., a paper sheet included in the kit), or machine-readable instructions (e.g., instructions carried on a magnetic or optical storage disk). In certain embodiments, machine-readable instructions comprise software for a programmable digital computer for comparing the measured values obtained using the reagents included in the kit.

In some embodiments, kits may also comprise a set of control values for one or more haptoglobin isoforms or variants thereof from a pooled sample from normal subjects (i.e., healthy subjects, or subjects without SCLC). These control values may be used to compare the level of haptoglobin markers from the tested sample to diagnosis the cancer or monitor the cancer progression.

Provided herein is also a device for obtaining data regarding a biological sample from a subject comprising a determination module configured to yield detectable signal from an assay indicating the presence or level of one or more haptoglobin isoforms or variants from the biological sample of a subject; and an output module for displaying an output content for a user. The device may further comprise a sample collection unit to hold the biological sample. The device may also comprise a storage module configured to store data output from the determination module; and a comparison assembly adapted to compare the data stored on the storage module with a control data, and to provide a retrieved data as the output content.

Alternatively, the device may further comprise a communication module for transmitting data from the determination module to an external device to compare with control data, and to transmitting a retrieved data back from the external device to the device as the output content. The present invention also provides the use of these devices to identify the likelihood of SCLC on a subject. In one embodiment, the device may be a handheld device, for example, a home use device.

In one embodiment, the data are stored in an external device, which may serve as a bioinformatics server, i.e., to store data bases including all the control data. In this regard, data read from the determination module may be transmitted to the external device through the communication module, and a retrieved data may be transmitted back from the external device to the device as the output content. Further, the transmitted data from measured assembly may be analyzed and the analyzed result may be transmitted back from the external device to the device as the output content.

The invention also provides a method of detecting small cell lung cancer (SCLC) in a subject, comprising the steps of: determining a level of one or more haptoglobin isoform, or fragment or variant thereof, in a biological sample of a subject; and comparing the level of the haptoglobin isoform, or fragment or variant thereof, in the biological sample of the subject with the level of the haptoglobin isoform, or fragment or variant thereof, in a control, wherein an elevated level of the haptoglobin isoform, or fragment or variant thereof, in the sample of the subject relative to the control indicates the subject may have SCLC.

The invention further provides a method of monitoring a subject at risk for developing SCLC, comprising the steps of: optionally obtaining a first biological sample from a subject at a first time point; optionally obtaining a second biological sample at a later time point; and determining the levels of one or more haptoglobin isoform, or fragment or variant thereof, in the first and second biological samples obtained at different times, wherein an increase in the level of the haptoglobin isoform, or fragment or variant thereof, in the second biological sample compared with the first biological sample indicates that the subject is at risk for developing SCLC.

The invention still further provides a method of monitoring the effect of a medical treatment or a medication on a subject being treated for SCLC, comprising the steps of: optionally obtaining a first biological sample from a subject at a first time point, optionally obtaining a second biological sample at a later time point, wherein the subject is exposed to a medical treatment or medication on or after the first time point; and determining the levels of one or more haptoglobin isoform, or fragment or variant thereof, in the first and second biological samples obtained before and after medical treatment or medication, wherein a decrease in the level of the haptoglobin isoform, or fragment or variant thereof, in the second biological sample compared with the first biological sample indicates an effective medical treatment or medication for the subject for treating SCLC.

In some aspects of all the methods, the haptoglobin isoform, or fragment or variant comprises at least one β haptoglobin isoform.

In some aspects of all the methods, the haptoglobin isoform, or fragment or variant comprises at least one haptoglobin minor isoform.

In some aspects of all the methods, the haptoglobin isoform, or fragment or variant comprises at least two haptoglobin minor isoforms.

In some aspects of all the methods, the haptoglobin minor isoform has a molecular weight of about 43 kDA to 44 kDa, and a basic pI.

In some aspects of all the methods, the haptoglobin minor isoform has a molecular weight of about 40 kDa, and an acidic pI.

The invention provides a method of detecting SCLC in a subject, comprising the step of: detecting the expression of one or more haptoglobin minor isoforms in a biological sample of the subject, wherein the haptoglobin minor isoform has a molecular weight of about 40 kDa, and an acidic pI; wherein the expression of the one or more haptoglobin minor isoforms in the biological sample of the subject indicates that the subject has SCLC.

The invention provides a method of detecting SCLC in a subject, comprising the steps of: optionally obtaining a biological sample from a subject; enriching at least one or more haptoglobin minor isoforms in the biological sample of the subject; detecting the expression of one or more haptoglobin minor isoforms in a biological sample of a subject, wherein the haptoglobin minor isoform has a molecular weight of about 40 kDa, and an acidic pI, wherein the expression of the one or more haptoglobin minor isoforms in the biological sample of the subject indicates that the subject has SCLC.

In some aspects of all the methods, the enriching haptoglobin isoforms comprises removing albumin, removing immunoglobulin, or both.

In some aspects of all the methods, the level of haptoglobin isoforms, or fragment or variant thereof, is measured by a protein-binding moiety.

In some aspects of all the methods, the protein binding moiety is selected from the group consisting of antibodies, recombinant antibodies, chimeric antibodies, tribodies, midibodies, protein-binding agents, small molecule, recombinant protein, peptides, aptamers, avimers and derivatives or fragments thereof.

In some aspects of all the methods, the protein binding moiety is an antibody comprising a polyclonal or a monoclonal anti haptoglobin antibody.

In some aspects of all the methods, the level of the haptoglobin isoform, or fragment or variant thereof, is measured using at least one method selected from the group consisting of immunoblot analysis, immunohistochemical analysis, radioimmunoassay (RIA), immunoradiometric assays, immunofluorescent assay, chemiluminescent assay, enzyme immunoassay, enzyme-linked immunosorbent assay (ELISA), sandwich immunoassays, gel diffusion precipitation reactions, immunodiffusion assays, in situ immunoassays, precipitation reactions, agglutination assays, complement fixation assays, isoform-specific chemical or enzymatic cleavage, protein array, protein A assays, immunoelectrophoresis assays, immuno-PCR, and mass spectrometry.

In some aspects of all the methods, the level of the haptoglobin isoform, or fragment or variant thereof is determined using a nucleic acid.

In some aspects of all the methods, the nucleic acid is mRNA.

In some aspects of all the methods, the biological sample is selected from the group consisting of: a blood sample, a plasma sample, a serum sample, a tissue sample, a tumor sample, a biopsy sample, an ex vivo cultivated sample, a ex vivo cultivated tumor sample, a surgically dissected tissue sample, a cancer sample, a lymph fluid sample, a primary ascite sample.

In some aspects of all the methods, the subject is a human subject.

The invention further provides a device for obtaining data regarding a biological sample from a subject comprising: a determination module configured to yield detectable signal from an assay indicating the presence or level of one or more haptoglobin isoform, or fragment or variant thereof from the biological sample of a subject; and an output module for displaying an output content for a user.

In some aspects of all the devices the device further comprises a sample collection unit to hold the biological sample.

In some aspects of all the devices the device further comprises: a storage module configured to store data output from the determination module; and a comparison assembly adapted to compare the data stored on the storage module with a control data, and to provide a retrieved data as the output content.

In some aspects of all the devices the device further comprises a communication module for transmitting data from the determination module to an external device to compare with control data, and to transmitting a retrieved data back from the external device to the device as the output content.

The invention also provides use of any one of the described devices to identify the likelihood of SCLC on a subject, wherein an elevated level of the haptoglobin isoform, or fragment or variant thereof in the sample of the subject compared to the control from the output module indicates that the subject is likely to have SCLC.

The invention further provides a use for any of the described devices for identifying the likelihood of SCLC on a subject, wherein the presence of the haptoglobin minor isoforms in the sample of the subject from the output module indicates that the subject is likely to have SCLC, and wherein the haptoglobin minor isoform has a molecular weight of about 40 kDa, and an acidic pI.

The invention provides a kit comprising: at least one reagent having specific affinity to one or more haptoglobin isoform, or fragment or variant thereof; and instructions for carrying out the method of any one of claims 1 to 19. The kit can further comprise a set of control values to the one or more haptoglobin isoform, or fragment or variant thereof.

### Example

### Example 1

### Serum Sample Collection

Serum from control (pooled serum from normal individuals) or SCLC patients (eight patients representing stage III and stage IV) were collected as described in the literature (Brundage, et al., 122 Chest 1037-57 (2002)), with approved informed consent from institutional review board.

### Serum Haptoglobin Enrichment by Albumin and Immunoglobulin Depletion

The method reported elsewhere was followed with modifications to remove albumin. Colantonio et al., 2005. Briefly, serum samples of 100 to 500 μl were incubated with 0.1 M NaCl for 1 hr at 4° C. Cold ethanol was added to the sample to reach a final concentration of ethanol at 42%. The mixture was incubated at 4° C. for 1 hr and then centrifuged at 16,000 g for 45 min. The pellet and supernatant were retained in separate micro tubes.

The first pellet was washed in 42% ethanol and centrifuged at 16,000 g for 45 min. The resulting second pellet was retained, and the resulting second supernatant discarded. The pH of the first supernatant collected after ethanol precipitation was reduced to 5.7 using cold 0.8 M sodium acetate (pH 4.0), and incubated at 4° C. for 1 hr. This mixture was then centrifuged at 16,000 g for 45 min. Once again, the resulting pellet and supernatant centrifuged from the first supernatant were retained in separate micro tubes. The retained two supernatant with albumin were pooled and used for analytical purposes.

The retained two pellets were combined and re-suspended in a triton-X lysis buffer. The re-suspended pellets were then further processed in order to remove immunoglobulins using iron nanoparticles. Briefly, protein A coated-iron nanoparticles (Miltenyi Biotec, Auburn, Calif.) were added to the albumin-free serum sample protein mixture and incubated at room temperature for 1 hr. A microcolumn was equilibrated with 200 μl lysis buffer. The sample mixture with protein A coated nanoparticles was then loaded into the column and allowed to elute. Immunoglobulins in the sample attached to nanoparticles, which were retained within the magnetic column, and the immunoglobulin-free protein sample was then obtained.

### Two Dimensional Gel Electrophoresis (2D-GE)

Tri-Chloro Acetic Acid (TCA)/Acetone Precipitation: The immunoglobulin-free protein sample was subject to the TCA/Acetone precipitation for 2D-GE. TCA/Acetone precipitation was performed to optimize serum protein recovery. 100 μl of serum sample was mixed with 800 μl ice-cold acetone and 100 μl 100% TCA. Following incubation at −20° C. for 1 hr and centrifugation steps, the precipitated protein pellet was dissolved in 250 μl of two dimensional gel rehydration buffer (8 M urea, 2% (3-[(3-cholamidopropyl)dimethylammonio]-1-propanesulfonate) (CHAPS), 50 mM dithiothreitol (DTT) (Sigma-Aldrich, Inc., St. Louis, Mo.), and 0.2% BIO-LYTE®, AMPHOLINE™ (Bio-Rad Laboratories, Inc., Hercules, Calif.).

Serum Protein Quantification: To achieve equal loading for 2D-GE, the protein concentration of the samples solubilized in the rehydration buffer were determined using a RC DC assay kit (Bio-Rad Laboratories, manufacturer's protocol was followed).

Rehydration of Immobilized pH gradient (IPG) Strip for 2D Electrophoresis: A known amount of protein was diluted with rehydration buffer (8M urea, 2% CHAPS, 50 mM DTT, and 0.2% BIO-LYTE®, AMPHOLINE™). The IPG strips (Bio-Rad Laboratories) with pI 4-pI 7 were rehydrated with 125 μl of the diluted sample for 11-16 hr.

First Dimensional Analysis Using Isolelectric Focusing (IEF): IEF was done using a PROTEAN® IEF Cell (Bio-Rad Laboratories), with the following protocol: 100 V for 3 hr, 300 V for 2 hr, 600 V for 1 hr, 1000 V for 1 hr, 2000 V for 1 hr, and 3000 V for 8 hr.

Second Dimensional Analysis by sodium dodecyl sulfate polyacrylamide gel electrophoresis (SDS-PAGE): Following the IEF, the IPG strips were equilibrated in two different buffers. The first equilibration buffer is composed of 6M urea, 0.375 M Tris-HCl, pH 8.8, 2% SDS, 20% glycerol and 2% DTT; and the second equilibration buffer has similar components as the first equilibration buffer except that it has 2.5% iodoacetamide instead of DTT. The IPG strips were equilibrated for 20 min in each equilibration buffer. The proteins were then further fractionated on SDS-PAGE gel and were analyzed either by immunoblot analysis or by silver staining.

Immunoblotting with Anti-Haptoglobin Antibody

To perform an immunoblot analysis for haptoglobin, the serum samples were analyzed by 12.5% SDS-PAGE and immunoblotting was performed with anti haptoglobin antibody (Sigma-Aldrich). Results were visualized using chemiluminescent reagents (PerkinElmer Inc, Waltham, Mass.) and autoradiography.

### Mass Spectrometry

Proteins from the silver stained gel were excised and processed for in-gel digestion as described in the literature. Rosenfeld et al., 203 Anal. Biochem. 173-79 (1992); Wilm et al., 68 Anal. Chem. 1-8 (1996). Briefly, gels were cut into small, uniform pieces; the gel pieces were dehydrated by acetonitrile and then rehydrated by 100 mM ammonium bicarbonate. Samples were treated with 10 mM DTT to reduce disulphide bonds followed by 50 mM iodoacetamide to block disulfides from reforming. Gel pieces were then washed with ammonium bicarbonate and dried by acetonitrile and the washing and drying steps were repeated twice. After completely dehydration using acetonitrile, gel pieces were suspended in 12.5 ng/μl trypsin in 50 mM ammonium bicarbonate. In-gel digestion was carried at 37° C. for 10-12 hr. The peptides were extracted from the gel in 50% acetonitrile and 5% formic acid. The extract was concentrated under reduced pressure and finally desalted by C18 containing ZIPTIP® (Millipore, Billerica, Mass.). Trypsin digested peptides were analyzed by Electrospray Ionization Mass Spectrometry (ES-MS/MS) and Matrix-Assisted Laser Desorption/Ionization Time-Of-Flight Mass Spectrometer (MALDI-TOF/MS), 4800 MALDI-TOF/TOF (Applied Biosystems, Inc, Foster City, Calif.)

MALDI-TOF/TOF MS analyses were performed using the following parameters: source 1 voltage at 80 kV, source 2 voltage at 15 kV, source 1 focus at 4.3 kV, source 1 lens at 3.7 kV, Y1 deflector at 0.08 kV, grid source 1 voltage ration of 0.91, mirror 2 to minor 1 voltage ratio of 1.7, and minor 2 to source 2 voltage ratio of 1.23. MS spectra were recorded in a mass range of 800-4000 Daltons. Data were collected and analyzed using the EXPLORER® software package (Applied Biosystems).

Mass spectrometry data were analyzed and interpreted using the Mascot™ version 2.0.04 (Matrix Science Ltd, London, UK), and ProteinProspector versions 4.27.2 and 5.0 (University of California, San Francisco, Calif.) database search programs. The database search parameters were as follows: the NCBInr database, SwissProt, tryptic digest, 2 missed cleavages, and a mass tolerance of 20-50 ppm. MS/MS data was analyzed by Mascot.

### Example 2

### Differentially Expressed Haptoglobin Isoforms in SCLC Patient Serum

To determine the differential expression of specific Haptoglobin isoforms, serum samples were fractionated by 2D-GE and various haptoglobin isoforms were estimated quantitatively by immunoblot analysis with anti-haptoglobin antibody. Serum samples from control (pooled serum from normal individuals) and SCLC patients (eight patients representing stage III and stage IV of SCLC) were acetone precipitated and equal amount of proteins were analyzed.

The immunoblot analysis with anti haptoglobin antibody demonstrated the presence of β, α-1 and α-2 Haptoglobin chains in control and as well as in SCLC patients. Both α and β isoforms expressed elevated levels in SCLC patient samples. The samples presented thick haptoglobin bands, indicating strong signals of haptoglobin expression, at 45 kDa (β chain), 19 kDa (α-2 chain) and 9 kDa (α-1 chain) (FIGS. 1A and 1B). A comparative analysis of SCLC serum samples with respect to the control serum samples showed presence of a set of three spots, each of them about 5 kDa smaller than haptoglobin 13-chain at pIs ranging from pI 5 to pI 6 (FIG. 1B, marked a, b, c, d). These haptoglobin minor isoforms, however, were not visualized in control serum samples. Quantitative estimations of these haptoglobin minor isoforms indicated the differences of expression levels between patients at stage III and IV of SCLC. The expression of these haptoglobin minor isoforms also varied between individual patients. (Table 1)

TABLE 1
Quantitative representation of different 40 kDa haptoglobin
isoforms in SCLC patient serum

| | Disease | Size at | | Size at | |
|---|---|---|---|---|---|
| Patient ID | Stage | Spot A | Size at Spot B | Spot C | Size at Spot D |
| Control | n/a | − | − | − | − |
| 120498 | IV | + | ++ | ++ | ++ |
| 1271205 | IV | + | +++ | +++ | +++ |
| 1360287 | IV | + | + | ++ | ++ |
| 1382081 | IIIB | + | + | +++ | +++ |
| 1394305 | IIIB | ++ | +++ | +++ | +++ |

### Haptoglobin Enrichment by Ethanol Precipitation

To further characterize these low abundance haptoglobin minor isoforms, haptoglobin was partially enriched by removing albumin and immunoglobulin. The steps of removal of albumin through ethanol/sodium acetate precipitation and protein A based removal of immunoglobulin were combined and optimized.

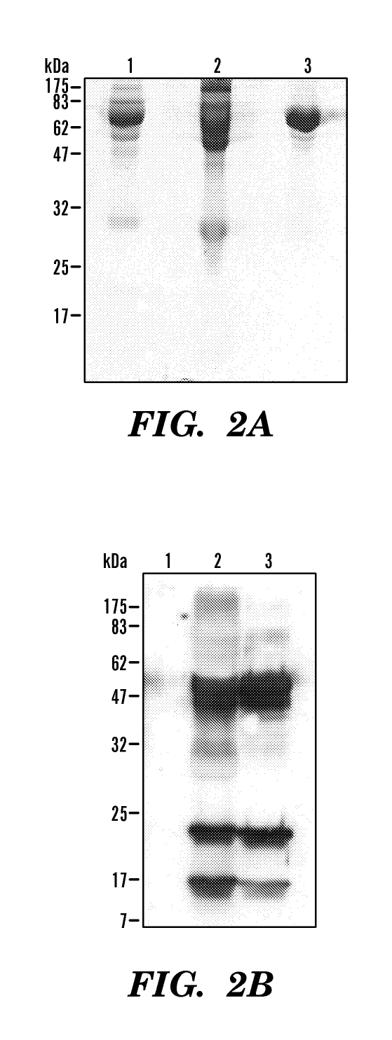

The haptoglobin-enriched samples were analyzed by SDS-PAGE and coomassie stain. FIG. 2A clearly indicated a significant removal of IgG and albumin from the samples. Most of the albumin was removed and collected as separate fractions. To determine the loss of haptoglobin during fractionation, various albumin fractions post-removal were analyzed by immunoblot analysis with an anti-haptoglobin antibody. Immunoblot analysis of post-enrichment fractions clearly demonstrated the complete absence of haptoglobin in the albumin fractions, as shown in FIG. 2B. All three major isoforms (β chain: 45 kDa; α-2 chain: 19 kDa and α-1 chain: 9 kDa) in the haptoglobin-enriched samples were visualized (FIG. 2B).

### Two-Dimensional Gel Electrophoresis (2D-GE) Analysis of Haptoglobin-Enriched Serum

To fractionate and identify different haptoglobin isoforms, haptoglobin-enriched serum samples were analyzed by 2D-GE, followed by second dimensional analysis by sodium dodecyl sulfate polyacrylamide gel electrophoresis (SDS-PAGE). To visualize the proteins, the SDS-PAGE was subjected to silver staining.

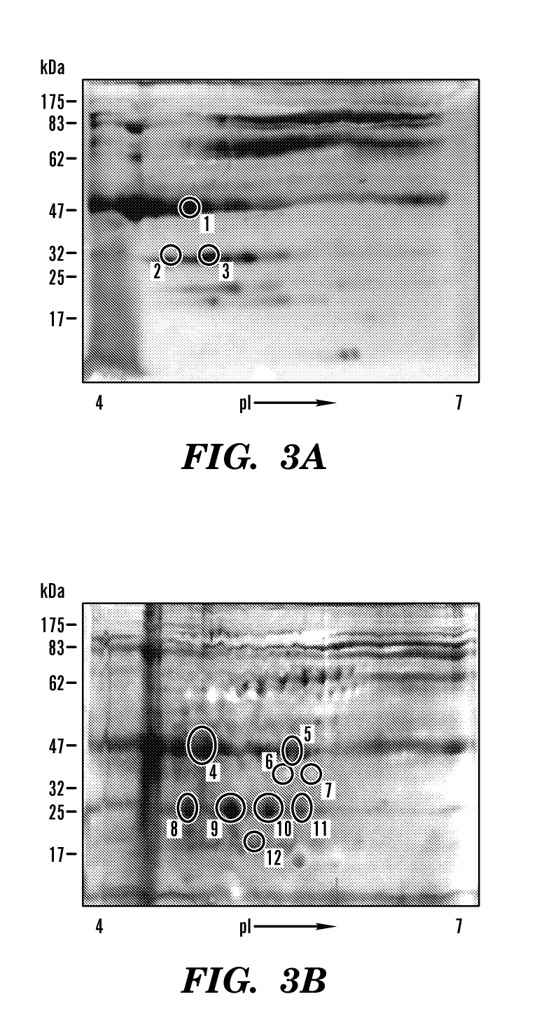

2D-GE of haptoglobin-enriched control serum samples showed a streaking pattern of three major proteins: albumin (64 kDa), the immunoglobulin heavy chain, and both the α- and β-chains of haptoglobin, as shown in FIG. 3A. Several silver stain protein spots of the gel were excised and analyzed by MS for protein identification.

### Characterization of Different Haptoglobin Isoforms

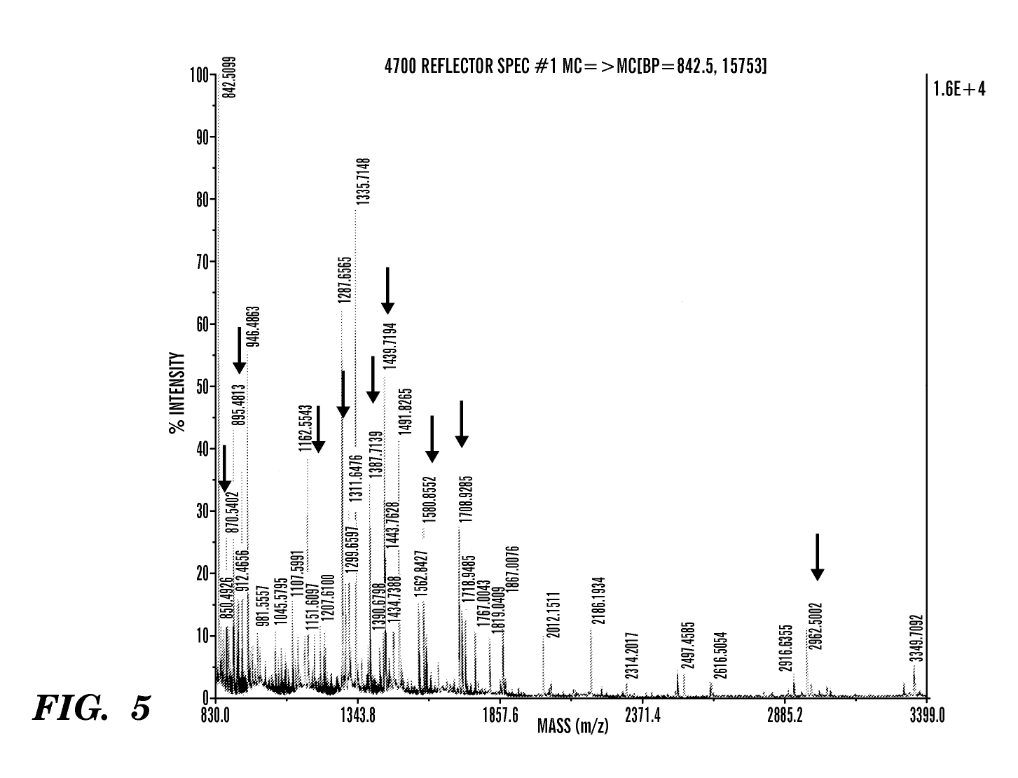

The 2D-GE of haptoglobin-enriched SCLC patient serum samples showed a streaking pattern with spots within the streaks. Haptoglobin streaks/spots were visualized at 45 kDa, 19 kDa and 9 kDa representing β, α-2 and α-1 isoforms. Characterization of protein streaks and spots at 45 kDa identified several haptoglobin isoforms as well as two apolipoproteins with similar mass and pIs, shown in FIG. 3B and Table 2. The pI of these β isoforms ranged from pI 5.5 to pI 6.5. Four haptoglobin isoforms of α-2 with masses of approximately 19 kDa were also identified. The pI range of the α-2 isoforms varied from pI 4.5 to pI 6.5 (FIG. 3B). Two haptoglobin isoforms of α-1 with a molecular mass of 9 kDa were identified by MS analysis in control and patient serum samples (FIGS. 3A and B). The protein identification was based on ES-MS/MS or MALDI-TOF/TOF/MS analysis; and to confirm the identification of the proteins, at least four representative peptides and at least two representative peptide MS/MS were considered. The major haptoglobin isoforms were represented by several peptides and MS/MS data of most of the peptides showed significant y and b ion fragments (FIG. 5).

The protein sequences referred to in Table 2 include:

(P00739; SEQ ID NO: 1)
msdlgavisl llwgrqlfal ysgndvtdis ddrfpkppei angyvehlfr yqcknyyrlr
tegdgvytln dkkqwinkav gdklpeceav cgkpknpanp vqrilgghld akgsfpwqak
mvshhnlttg atlineqwll ttaknlflnh senatakdia ptltlyvgkk qlveiekvvl
hpnyhqvdig liklkqkvlv nervmpiclp sknyaevgrv gyvsgwgqsd nfkltdhlky
vmlpvadqyd cithyegstc pkwkapkspv gvqpilneht fcvgmskyqe dtcygdagsa
favhdleedt wyaagilsfd kscavaeygv yvkvtsiqhw vqktiaen;
(P00738; SEQ ID NO: 2)
msalgavial llwgqlfavd sgndvtdiad dgcpkppeia hgyvehsvry qcknyyklrt
egdgvytlnd kkqwinkavg dklpeceadd gcpkppeiah gyvehsvryq cknyyklrte
gdgvytlnne kqwinkavgd klpeceavcg kpknpanpvq rilgghldak gsfpwqakmv
shhnlttgat lineqwlltt aknlflnhse natakdiapt ltlyvgkkql veiekvvlhp
nysqvdigli klkqkvsvne rvmpiclpsk dyaevgrvgy vsgwgrnanf kftdhlkyvm
lpvadqdqci rhyegstvpe kktpkspvgv qpilnehtfc agmskyqedt cygdagsafa
vhdleedtwy atgilsfdks cavaeygvyv kvtsiqdwvq ktiaen
(P02647; SEQ ID NO: 3)
mkaavltlav lfltgsqarh fwqqdeppqs pwdrvkdlat vyvdvlkdsg rdyvsqfegs
algkqlnlkl ldnwdsvtst fsklreqlgp vtqefwdnle keteglrqem skdleevkak
vqpylddfqk kwqeemelyr qkveplrael qegarqklhe lqeklsplge emrdrarahv
dalrthlapy sdelrqrlaa rlealkengg arlaeyhaka tehlstlsek akpaledlrq
gllpvlesfk vsflsaleey tkklntq;
(119579598; SEQ ID NO: 4)
mphstalpea rptkmsalga vialllwgql favdsgndvt diaddgcpkp peiahgyveh
svryqcknyy klrtegdgvy tlnnekqwin kavgdklpec eaddgcpkpp eiahgyvehs
vryqcknyyk lrtegdgvyt lndkkqwink avgdklpece avcgkpknpa npvqrilggh
ldakgsfpwq akmvshhnlt tgatlineqw llttaknlfl nhsenatakd iaptltlyvg
kkqlveiekv vlhpnysqvd igliklkqkv svnervmpic lpskdyaevg rvgyvsgwgr
nanfkftdhl kyvmlpvadq dqcirhyegs tvpekktpks pvgvqpilne htfcagmsky
qedtcygdag safavhdlee dtwyatgils fdkscavaey gvyvkvtsiq dwvqktiaen;
(Q9BWW8; SEQ ID NO: 5)
mdnqaerese agvglqrded daplcedvel qdgdlspeek iflrefprlk edlkgnidkl
raladdidkt hkkftkanmv atstavisgv msllglalap atgggsllls tagqglataa
gvtsivsgtl ersknkeaqa raedilptyd qedredeeek adyvtaagki iynlrntlky
akknvrafwk lranprlana tkrllttgqv ssrsrvqvqk afagttlamt knarvlggvm
safslgydla tlskewkhlk egartkfaee lrakaleler klteltqlyk slqqkvrsra
rgvgkdltgt ceteaywkel rehvwmwlwl cvclcvcvyv qft.

The proteins or proteins fractions of the proteins set forth herein can be used to make antibodies, either polyclonal or monoclonal according to routine methods well known to one skilled in the art.

TABLE 2

| | | | Molecular | | | |
|---|---|---|---|---|---|---|
| Spot | Protein | Accession | Weight | | | SEQ ID |
| Number | Identification | Number | (kDa) | pI | Function | NO: |
| 1 | Haptoglobin- | P00739 | 39.0 | 6.4 | Acute-Phase | 1 |
| | Related Protein | | | | Protein | |
| | Precursor | | | | | |
| 2 | Haptoglobin | P00738 | 45.2 | 6.1 | Acute-Phase | 2 |
| | Precursor | | | | Protein | |
| 3 | Haptoglobin | P00738 | 45.2 | 6.1 | Acute-Phase | 2 |
| | Precursor | | | | Protein | |
| 4 | Haptoglobin- | P00739 | 39.0 | 6.4 | Acute-Phase | 1 |
| | Related Protein | | | | Protein | |
| 5 | Apolipoprotein | P02647 | 30.7 | 5.6 | Primary Protein | 3 |
| | A-1 Precursor | | | | Constituent of | |
| | | | | | HDL | |
| 6 | Haptoglobin | P00738 | 45.2 | 6.1 | Acute-Phase | 2 |
| | Precursor | | | | Protein | |
| 7 | Apolipoprotein | Q9BWW8 | 38.4 | 8.6 | Acute-Phase | 5 |
| | L-6 | | | | Protein | |
| 8 | Haptoglobin | P00738 | 45.2 | 6.1 | Acute-Phase | 2 |
| | Precursor | | | | Protein | |
| 9 | Haptoglobin | P00738 | 45.2 | 6.1 | Acute-Phase | 2 |
| | Precursor | | | | Protein | |
| 10 | Haptoglobin, | 119579598 | 46.7 | 6.3 | Acute-Phase | 4 |
| | Isoform CRA_a | | | | Protein | |
| 11 | Haptoglobin, | 119579598 | 46.7 | 6.3 | Acute-Phase | 4 |
| | Isoform CRA_a | | | | Protein | |
| 12 | Haptoglobin- | P00739 | 39.0 | 6.4 | Acute-Phase | 1 |
| | Related Protein | | | | Protein | |
| | Precursor | | | | | |

Haptoglobin Isoforms Differentially Expressed in SCLC Patient Serum

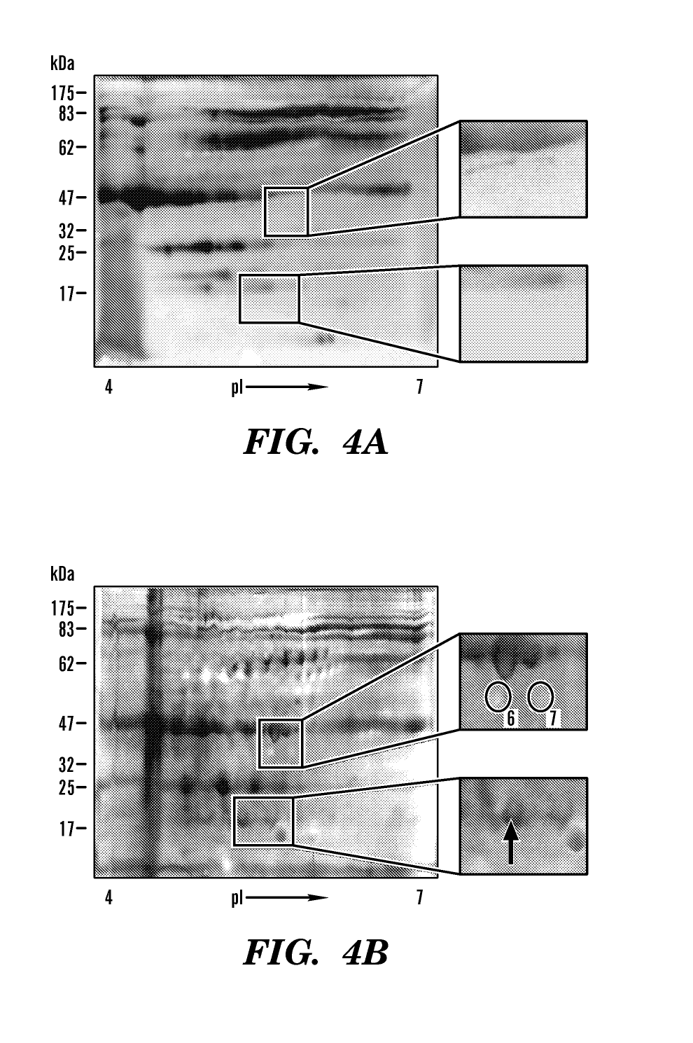

In a series of experiments, haptoglobin-enriched serum samples were fractionated by 2D-GE to identify and characterize differentially expressed haptoglobin isoforms. Haptoglobin-enriched serum samples showed some similarity between control and SCLC samples. A comparative analysis of control and SCLC serum samples by 2D-GE and silver stain indicated two surprising differences: higher level of haptoglobin in SCLC sample compared with control sample; and the presence of 40 kDa minor haptoglobin isoforms in SCLC samples only. As shown in FIG. 4A and FIG. 4B, SCLC patient sera showed an elevated level of haptoglobin compared to control sera; and only SCLC patent sera contain two 40 kDa isoforms of the β chain. Differentially expressed 40 kDa isoforms were negatively stained with silver. One of the spots was identified as haptoglobin by MS analysis. These proteins also cross reacted with anti-haptoglobin antibody (FIG. 1B, spot c).

1. An assay comprising the steps of:
determining a level of one or more haptoglobin isoform, or fragment or variant thereof, in a biological sample of a subject; and
comparing the level of the haptoglobin isoform, or fragment or variant thereof, in the biological sample of the subject with a reference,
wherein an elevated level of the haptoglobin isoform, or fragment or variant thereof, in the sample of the subject relative to the reference indicates the subject may have small cell lung cancer (SCLC).

2. The assay of claim 1, wherein the haptoglobin isoform, or fragment or variant comprises at least one β haptoglobin isoform.

3. The assay of claim 1, wherein the haptoglobin isoform, or fragment or variant comprises at least one haptoglobin minor isoform.

4. The assay of claim 3, wherein the haptoglobin isoform, or fragment or variant comprises at least two haptoglobin minor isoforms.

5. The assay of claim 3, wherein the haptoglobin minor isoform has a molecular weight of about 43 kDA to 44 kDa, and a basic pI.

6. The assay of claim 4, wherein the haptoglobin minor isoform has a molecular weight of about 40 kDa, and an acidic pI.

7. The assay of claim 1, wherein the level of haptoglobin isoforms, or fragment or variant thereof, is measured by a protein-binding moiety.

8. The assay of claim 1, wherein the level of the haptoglobin isoform, or fragment or variant thereof is determined using a nucleic acid.

9. An assay comprising the steps of:
determining the levels of one or more haptoglobin isoform, or fragment or variant thereof, in a first biological sample obtained from a subject at a first time point and a second biological sample taken from the subject at a second time point,
comparing the haptoglobin isoform, or fragment or variant thereof level in the first biological sample to the level of haptoglobin isoform, or fragment or variant thereof in the second biological sample,
wherein an increase in the level of the haptoglobin isoform, or fragment or variant thereof, in the second biological sample compared with the first biological sample indicates that the subject is at risk for developing small cell lung cancer (SCLC).

10. The assay of claim 9, wherein the haptoglobin isoform, or fragment or variant comprises at least one β haptoglobin isoform.

11. The assay of claim 9, wherein the haptoglobin isoform, or fragment or variant comprises at least one haptoglobin minor isoform.

12. The assay of claim 11, wherein the haptoglobin isoform, or fragment or variant comprises at least two haptoglobin minor isoforms.

13. The assay of claim 11, wherein the haptoglobin minor isoform has a molecular weight of about 43 kDA to 44 kDa, and a basic pI.

14. The assay of claim 12, wherein the haptoglobin minor isoform has a molecular weight of about 40 kDa, and an acidic pI.

15. The assay of claim 11, wherein the level of haptoglobin isoforms, or fragment or variant thereof, is measured by a protein-binding moiety.

16. The assay of claim 9, wherein the level of the haptoglobin isoform, or fragment or variant thereof is determined using a nucleic acid.

17. An assay comprising the steps of:
determining level of one or more haptoglobin isoform, or fragment or variant thereof, in a first biological sample obtained from a subject diagnosed with small cell lung carcinoma (SCLC) at a first time point and in a second biological sample obtained from the subject at a later time point, wherein the subject is exposed to a medical treatment or medication between the first and the later time point,
comparing the level of one or more haptoglobin isoform, or fragment or variant thereof between the first and the later time point,
wherein a decrease in the level of the haptoglobin isoform, or fragment or variant thereof, in the sample obtained at the later time point compared with the first time point indicates an effective medical treatment or medication for the subject for treating SCLC.

18. The assay of claim 17, wherein the haptoglobin isoform, or fragment or variant comprises at least one β haptoglobin isoform.

19. The assay of claim 17, wherein the haptoglobin isoform, or fragment or variant comprises at least one haptoglobin minor isoform.

20. The assay of claim 14, wherein the haptoglobin isoform, or fragment or variant comprises at least two haptoglobin minor isoforms.

21. The assay of claim 16, wherein the haptoglobin minor isoform has a molecular weight of about 43 kDA to 44 kDa, and a basic pI.

22. The assay of claim 16, wherein the haptoglobin minor isoform has a molecular weight of about 40 kDa, and an acidic pI.

23. The assay of claim 17, wherein the level of haptoglobin isoforms, or fragment or variant thereof, is measured by a protein-binding moiety.

24. The assay of claim 17, wherein the level of the haptoglobin isoform, or fragment or variant thereof is determined using a nucleic acid.

25. An assay, comprising the step of:
detecting the expression of one or more haptoglobin minor isoforms in a biological sample obtained from a subject, wherein the haptoglobin minor isoform comprises an isoform that has a molecular weight of about 40 kDa, and an acidic pI;
wherein the expression of the an isoform that has a molecular weight of about 40 kDa, and an acidic pI in the biological sample indicates that the subject has small cell lung carcinoma (SCLC).

26. An assay comprising the steps of:
enriching at least one or more haptoglobin minor isoforms in a biological sample obtained from a subject;
detecting the expression of one or more haptoglobin minor isoforms in a biological sample of a subject, wherein the one or more haptoglobin minor isoforms comprises a haptoglobin minor isoform that has a molecular weight of about 40 kDa, and an acidic pI,
wherein presence of the expression of the haptoglobin minor isoform that has a molecular weight of about 40 kDa, and an acidic pI indicates that the subject has small cell lung carcinoma.

27. The method of claim 26, wherein enriching haptoglobin isoforms comprises removing albumin, removing immunoglobulin, or removing both albumin and immunglobin from the biological sample prior to the stem of detecting.

28. A device for obtaining data regarding a biological sample from a subject comprising:
a determination module configured to yield detectable signal from an assay indicating the presence or level of one or more haptoglobin isoform, or fragment or variant thereof from the biological sample of a subject; and
an output module for displaying an output content for a user.

29. The device of claim 28, further comprising a sample collection unit to hold the biological sample.

30. The device of claim 29, further comprising:
a storage module configured to store data output from the determination module; and
a comparison assembly adapted to compare the data stored on the storage module with a control data, and to provide a retrieved data as the output content.

31. The device of claim 30, further comprising a communication module for transmitting data from the determination module to an external device to compare with control data, and to transmitting a retrieved data back from the external device to the device as the output content.
